(19) United States
(12) Patent Application Publication
(10) Pub. No.: US 2026/0125636 A1
(43) Pub. Date: May 7, 2026

(54) LIMOSILACTOBACILLUS REUTERI SUBSP. REUTERI LR1349, AND COMPOSITION INCLUDING THE SAME AND USE THEREOF

(71) Applicant: SYNBIO TECH INC., Kaohsiung City (TW)

(72) Inventors: Chi-Hao WU, Taipei City (TW); Han-Yin HSU, Kaohsiung City (TW); Chia-Chia LEE, Kaohsiung City (TW); Jin-Seng LIN, Tainan City (TW)

(21) Appl. No.: 19/289,395

(22) Filed: Aug. 4, 2025

Related U.S. Application Data
(63) Continuation of application No. PCT/CN2025/101757, filed on Jun. 18, 2025.
(60) Provisional application No. 63/716,916, filed on Nov. 6, 2024.

(51) Int. Cl. C12N 1/20; A61K 35/00; A61P 27/02

(52) U.S. Cl. CPC C12N 1/205; A61P 27/02; A61K 2035/115

(57) ABSTRACT

Limosilactobacillus reuteri subsp. reuteri LR1349 is deposited at the Deutsche Sammlung von Mikroorganismen und Zellkulturen (DSMZ) GmbH under an accession number DSM 35135 in accordance with the Budapest Treaty. A method for alleviating blue light-induced retinal damage using a composition containing Limosilactobacillus reuteri subsp. reuteri LR1349 is also provided. The composition is a food product or a pharmaceutical composition.

### CROSS-REFERENCE TO RELATED APPLICATION

This application is a continuation application of International Application No. PCT/CN2025/101757, filed on Jun. 18, 2025, which claims priority to U.S. Provisional Patent Application No. 63/716,916, filed on Nov. 6, 2024. The aforesaid applications are incorporated by reference herein in their entirety.

### SEQUENCE LISTING XML

The Sequence Listing submitted concurrently herewith with a file name of “PE-72139-AM-SEQUENCE_LISTING.xml,” a creation date of Jun. 26, 2025, and a size of 13,974 bytes, is part of the specification and is incorporated by reference in its entirety.

### FIELD

The disclosure relates to Limosilactobacillus reuteri subsp. reuteri LR1349, and a method for alleviating blue light-induced retinal damage using the same.

### BACKGROUND

Blue light is a high-energy visible light, and exposure to blue light too frequently may cause a retina of an eye to be damaged (i.e., retinal damage).

At present, there is no clinically effective method to alleviate blue light-induced retinal damage. The only way to avoid such damage is to reduce exposure to blue light sources (e.g., computers, communication devices, and consumer electronics), and to consume health supplements that are beneficial to eyes, such as lutein and fish oil.

Lactic acid bacteria (LAB) are generally recognized as safe (GRAS), well-known and widely used probiotics, and have been found to have the effects of inhibiting the growth of gastrointestinal pathogens, alleviating lactose intolerance, anti-cancer and lowering blood pressure. There are many types of lactic acid bacteria that can be used as probiotics, such as Lactobacillus (part thereof is also known as Limosilactobacillus), Lactococcus, Pediococcus, Enterococcus, Streptococcus, and Bifidobacterium.

In spite of the aforesaid, there is still a need to develop a method that is effective in alleviation of blue light-induced retinal damage.

### SUMMARY

Accordingly, the present disclosure provides a method for alleviating blue light-induced damage, which can alleviate at least one of the drawbacks of the prior art, and which includes administering to a subject in need thereof a composition containing Limosilactobacillus reuteri subsp. reuteri LR1349, which is deposited at the Deutsche Sammlung von Mikroorganismen und Zellkulturen (DSMZ) GmbH under an accession number DSM 35135 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows genetic fingerprints of a conventional Limosilactobacillus reuteri subsp. reuteri DSM 20016^{T} (Lane 1) and Limosilactobacillus reuteri subsp. reuteri LR1349 (Lane 2) according to the present disclosure, infra, which are respectively obtained by random amplified polymorphic DNA (RAPD) analysis using different 10-mer primers, and in which Lane M represents a DNA ladder marker (100 bp to 3000 bp).

FIG. 2 shows the visual acuity threshold of the mice in each group of Example 2 determined at the end of each of the 8^{th} week and the 16^{th} week after starting irradiation with blue light, infra, in which the data of different groups in each of the 8^{th} week and the 16^{th} week are marked with different English letters to indicate statistical differences therebetween.

FIG. 3 shows the retinal potential changes of the mice in each group of Example 2 determined at the end of each of the 8^{th} week and the 16^{th} week after starting the irradiation with the blue light, infra, in which the data of different groups for each of the a-wave and the b-wave measured in each of the 8^{th} week and the 16^{th} week are marked with different English letters to indicate statistical differences therebetween.

FIG. 4 shows the result of histopathological analysis of the mice in each group of Example 2 determined at the end of the 16^{th} week after starting the irradiation with the blue light, infra, in which “GCL” represents a ganglion cell layer, “INL” represents an inner nuclear layer, “ONL” represents an outer nuclear layer, “IS/OS” represents an inner segment/outer segment layer, and “RPE” represents a retinal pigment epithelium.

FIG. 5 shows the average thickness of the ONL of the mice in each group of Example 2 determined at the end of the 16^{th} week after starting the irradiation with the blue light, infra, in which the data of different groups are marked with different English letters, to indicate statistical differences therebetween.

FIG. 6 shows the average thickness of the IS/OS of the mice in each group of Example 2 determined at the end of the 16^{th} week after starting the irradiation with the blue light, infra, in which the data of different groups are marked with different English letters to indicate statistical differences therebetween.

FIG. 7 shows the number of nuclei in the ONL of the mice in each group of Example 2 determined at the end of the 16^{th} week after starting the irradiation with the blue light, infra, in which the data of different groups are marked with different English letters to indicate statistical differences between these data.

FIG. 8 shows the relative fluorescence intensity detected in the retinal tissue sections of the mice in each group of Example 2 determined at the end of the 16^{th} week after starting the irradiation with the blue light, infra, in which the data of different groups are marked with different English letters to indicate statistical differences therebetween.

### DETAILED DESCRIPTION

It is to be understood that, if any prior art publication is referred to herein, such reference does not constitute an admission that the publication forms a part of the common general knowledge in the art.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of approaches to alleviate blue light-induced retinal damage, the applicant surprisingly found that a strain of Limosilactobacillus reuteri subsp. reuteri isolate, which was named “Limosilactobacillus reuteri subsp. reuteri LR1349” by the applicant, and deposited at the Deutsche Sammlung von Mikroorganismen und Zellkulturen (DSMZ) GmbH under an accession number DSM 35135, has been demonstrated through in vivo experiments to be capable of effectively alleviating decreased visual acuity, retinal dysfunction and retinopathy caused by blue light exposure, and hence are expected to have high potential to be developed as drugs for use in alleviation of blue light-induced retinal damage.

Therefore, the present disclosure provides Limosilactobacillus reuteri subsp. reuteri LR1349, which is deposited at the Deutsche Sammlung von Mikroorganismen und Zellkulturen (DSMZ) GmbH under an accession number DSM 35135 in accordance with the Budapest Treaty.

The present disclosure also provides a method for alleviating blue light-induced retinal damage, which includes administering to a subject in need thereof a composition containing Limosilactobacillus reuteri subsp. reuteri LR1349, which is deposited at the Deutsche Sammlung von Mikroorganismen und Zellkulturen (DSMZ) GmbH under an accession number DSM 35135 in accordance with the Budapest Treaty.

In certain embodiments, the term “alleviating blue light-induced retinal damage” means that at least one of the following therapeutic efficacy is achieved: enhancing visual acuity, improving retinal function, and alleviating retinopathy.

As used herein, the terms “administering” and “administration” can be interchangeably used, and refer to introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human. In other embodiments, the subject does not have dry eye syndrome.

According to the present disclosure, the Limosilactobacillus reuteri subsp. reuteri LR1349 may be live cells or dead cells, concentrated or non-concentrated, a liquid, a paste, a semi-solid, or a solid (e.g., a pellet, a granule, or a powder), and may be heat-inactivated, frozen, dried, or freeze-dried (e.g., may be in freeze-dried form or spray/fluid bed dried form). In an exemplary embodiment, the Limosilactobacillus reuteri subsp. reuteri LR1349 is in the form of live cells.

According to the present disclosure, the Limosilactobacillus reuteri subsp. reuteri LR1349 may be prepared into a bacterial liquid having a total bacterial concentration ranging from 1×10⁶ CFU/mL to 1×10⁹ CFU/mL. In an exemplary embodiment, the bacterial liquid has a total bacterial concentration of 3.3×10⁸ CFU/mL.

According to the present disclosure, the culture medium suitable for culturing the Limosilactobacillus reuteri subsp. reuteri LR1349 may be prepared using techniques well-known to those skilled in the art, or may be commercially available products, which may include, but are not limited to, MRS (De Man, Rogosa and Sharpe) broth, MRS broth supplemented with cysteine, and a mineral medium supplemented with glucose and soy peptone. In an exemplary embodiment, the culture medium is the MRS broth.

As used herein, the terms “culturing”, “fermentation” and “cultivation” may be used interchangeably.

The procedures and parameter conditions for cultivation are within the expertise and routine skills of those skilled in the art, and may be adjusted according to practical requirements.

According to the present disclosure, the cultivation may be carried out at a temperature ranging from 30° C. to 37° C. for a time period ranging from 16 hours to 18 hours. In an exemplary embodiment, the cultivation is carried out at 37° C. for the time period ranging from 16 hours to 18 hours.

According to the present disclosure, the composition may be formulated as a food product using a standard technique well known to one of ordinary skill in the art. For instance, the aforesaid composition may be directly added to an edible material, or may be utilized for preparing an intermediate composition (e.g., a food additive or a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, and dietary supplements.

According to the present disclosure, the composition may be prepared in the form of a pharmaceutical composition.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may be formulated into a dosage form suitable for oral administration or parenteral administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intralesional injection and sublingual administration.

According to the present disclosure, the dose and frequency of administration of the composition may vary depending on the following factors: the severity of the illness or disorder to be treated, routes of administration, and age, physical condition and response of the subject to be treated. In general, the composition may be administered orally or parenterally in a single dose or in several doses.

The present disclosure also provides the aforesaid composition for use in alleviation of blue light-induced retinal damage. The use includes administering to a subject in need thereof the aforesaid composition containing the Limosilactobacillus reuteri subsp. reuteri LR1349.

The present disclosure further provides use of the aforesaid composition in the manufacture of a medication or a food product in alleviation of blue light-induced retinal damage in a subject.

The disclosure will be further described by way of the following examples. However, it should be understood that the following examples are solely intended for the purpose of illustration and should not be construed as limiting the disclosure in practice.

### EXAMPLES

### General Experimental Materials:

### 1. Experimental Mice:

Male ICR mice (8 weeks old) used in the following experiments were purchased from BioLasco Taiwan Co., Ltd. All the experimental mice were individually housed in an animal room under the following laboratory conditions: an alternating 12-hour light and 12-hour dark cycle, a temperature maintained at 22° C.±2° C., a relative humidity maintained at 60%±10%. Furthermore, water and feed were provided ad libitum for all the experimental mice. All the experimental mice were kept for 2 weeks before conducting the experiments in order to adapt to the environment. All experimental procedures involving the experimental mice were approved by the Institutional Animal Care and Use Committees in China Taiwan Normal University, and were carried out in accordance with the Guide for the Care and Use of Laboratory Animals of National Institutes of Health (NIH).

### General Procedures:

### 1. Statistical Analysis:

The experimental data of all the test groups are expressed as mean±standard deviation (SD), and were analyzed using one-way analysis of variance (one-way ANOVA) followed by post hoc test, so as to evaluate the differences between the groups. Statistical significance is indicated by p<0.05.

### Example 1. Screening and Characterization of Limosilactobacillus Isolate LR1349

### A. Source and Isolation of Limosilactobacillus Isolate LR1349:

Feces were collected from healthy humans aged over 85 years old and had not used antibiotics for 3 months so as to serve as a sample source for isolation and screening of Lactobacillus. First, the feces were mixed with an appropriate amount of saline solution (added with 0.05% of L-Cysteine) to obtain a mixture, and then the mixture was subjected to homogenization using a homogenizer (Scientific Industries, Inc.; Model: Vortex-Genie 2). Next, the homogenate thus obtained was subjected to 10-fold serial dilution with a saline solution, so that 10⁻¹-fold to 10⁻⁶-fold diluents were obtained. Subsequently, 0.1 mL of each of the 10⁻¹-fold to 10⁻⁶-fold diluents was taken and then evenly spread on Difco Lactobacilli MRS agar, followed by cultivation in an incubator (37° C.) under an anaerobic condition for 5 days to 7 days. The colonies formed on the Difco Lactobacilli MRS agar were picked to obtain an isolated strain, i.e., Limosilactobacillus isolate LR1349.

### B. Preliminary Tests:

The Limosilactobacillus isolate LR1349 was subjected to Gram staining, and the results showed that the Limosilactobacillus isolate LR1349 was Gram-positive bacilli.

### C. Extraction of Genomic DNA:

The Limosilactobacillus isolate LR1349 was subjected to extraction of genomic DNA conducted generally according to the method described in Zhu, H et al. (1993), Nucleic Acids Res., 21 (22): 5279-5280, in which an extraction buffer (containing 100 mM Tris and 40 mM EDTA; pH 9.0), Mini-BeadBeater Glass Mill Beads (0.1 mm) (BioSpec Products; Cat. No. 11079101), and 500 μL of phenol-chloroform-isoamyl alcohol mixture (25:24:1, v/v/v) were used.

D. 16S Ribosomal DNA (rDNA) Sequence Analysis:

The resultant genomic DNA of the Limosilactobacillus isolate LR1349 obtained in Section C of this example, serving as a template, was subjected to polymerase chain reaction (PCR) performed using a designed primer pair specific for bacterial 16S rDNA gene shown in Table 1 under the reaction conditions shown in Table 2 below, thereby obtaining a PCR product (i.e., PCR-amplified 16S rDNA fragments) of the same.

TABLE 1

| | Nucleotide sequence |
|---|---|
| PCR primer pair | (5′→3′) |
| Forward primer | agagtttgatcctggctcag |
| 27F | (SEQ ID NO: 1) |
| Reverse primer | ggttaccttgttacgact |
| 1492R | (SEQ ID NO: 2) |

TABLE 2

| Reaction mix | Volume (μL) |
|---|---|
| Genomic DNA (10 ng/μL) | 1 |
| Forward primer 27F (10 μM) | 0.5 |
| Reverse primer 1492R (10 μM) | 0.5 |
| Fast-Run ™ 2X Tag Master Mix | 10 |
| (Protech, Cat. No. SA-TMM228) | |
| Deionized water | 8 |

Operation conditions: denaturation at 95° C. for 5 minutes, followed by 30 cycles of the following reactions: denaturation at 95° C. for 60 seconds, primer annealing at 50° C. for 60 seconds, and extension at 72° C. for 60 seconds; finally, elongation at 72° C. for 8 minutes

Thereafter, the PCR product was verified by sequencing analysis which was entrusted to Genomics BioSci & Tech Co., Ltd., so as to obtain a part of 16S rDNA sequence of the Limosilactobacillus isolate LR1349 (SEQ ID No: 3). By comparing the data in the NCBI's gene database, the part of the 16S rDNA sequence of the Limosilactobacillus isolate LR1349 is found to have 97% identity to a part of the 16S rDNA sequence of Limosilactobacillus reuteri subsp. reuteri.

In view of the aforesaid experimental results, the Limosilactobacillus isolate LR1349 of the present disclosure is identified as Limosilactobacillus reuteri subsp. reuteri. In order to confirm whether such Limosilactobacillus reuteri subsp. reuteri LR1349 is a novel Limosilactobacillus reuteri subsp. reuteri strain, the following experiment was conducted.

### E. Random Amplified Polymorphic DNA (RAPD) Analysis:

The RAPD analysis was generally performed with reference to Akopyanz N. et al. (1992), Nucleic Acids Res., 20:5137-5142. Briefly, the resultant genomic DNA of the Limosilactobacillus isolate LR1349 obtained in Section C of this example, serving as a template, was subjected to polymerase chain reaction (PCR) performed using eight 10-mer primers shown in Table 3 under the reaction conditions shown in Table 4 below.

TABLE 3

| Primer | Nucleotide sequence (5′→3′) |
|---|---|
| RAPD-1 | ctcaggtcgc (SEQ ID NO: 4) |
| RAPD-A | ccgcagccaa (SEQ ID NO: 5) |
| RAPD-B | aacgcgcaac (SEQ ID NO: 6) |
| RAPD-C | gcggaaatag (SEQ ID NO: 7) |
| RAPD-D | gaggacaaag (SEQ ID NO: 8) |
| RAPD-E | ggcgtcggtt (SEQ ID NO: 9) |
| RAPD-F | ggccacggaa (SEQ ID NO: 10) |
| RAPD-G | cgaactagac (SEQ ID NO: 11) |

TABLE 4

| Reaction mix | Volume (μL) |
|---|---|
| Genomic DNA (50-100 ng) | 1 |
| 10-mer primer (10 μM) | 4 |
| 2X Taq Master Mix (Ampliqon, 5200300-1250) | 12.5 |
| MgCl₂ (25 mM) | 2 |
| Deionized water | 5.5 |

Operation conditions: denaturation at 94° C. for 2 minutes, followed by 6 cycles of the following reactions: denaturation at 94° C. for 30 seconds, primer annealing at 36° C. for 60 seconds, and extension at 72° C. for 90 seconds, and then by 30 cycles of the following reactions: denaturation at 94° C. for 20 seconds, primer annealing at 36° C. for 30 seconds, and extension at 72° C. for 90 seconds; finally, elongation at 72° C. for 3 minutes

The resultant PCR product was subjected to 1.5% agarose gel electrophoresis analysis, followed by staining, and then observation and photography under ultraviolet light.

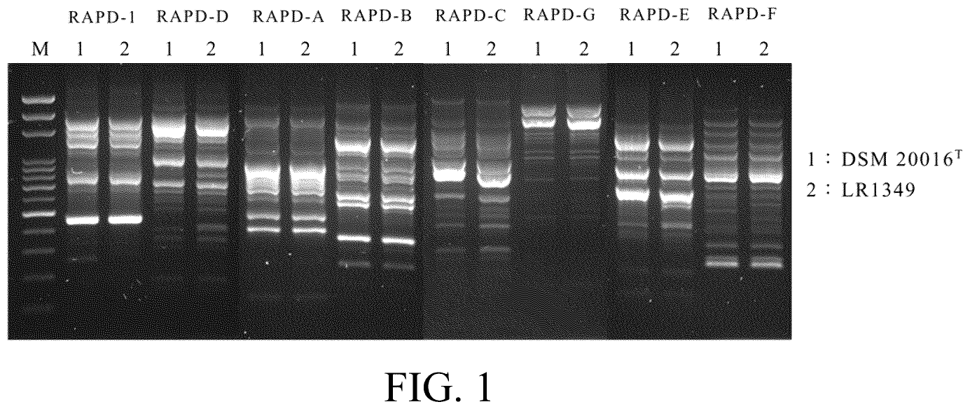

For comparison purpose, the applicant used a conventional Limosilactobacillus reuteri subsp. reuteri DSM 20016^{T} (corresponding to Limosilactobacillus reuteri subsp. reuteri BCRC 14625^{T}) as a comparative strain to be compared with the Limosilactobacillus reuteri subsp. reuteri LR1349. The results of the 1.5% agarose gel electrophoresis analysis were shown in FIG. 1.

The results in FIG. 1 show that the genetic fingerprints of the Limosilactobacillus reuteri subsp. reuteri LR1349 and the conventional Limosilactobacillus reuteri subsp. reuteri DSM 20016^{T} were different from each other.

Based on the aforementioned characterization results, the applicant believes that the Limosilactobacillus reuteri subsp. reuteri LR1349 of the present disclosure is a novel strain of Limosilactobacillus reuteri subsp. reuteri. As such, the Limosilactobacillus reuteri subsp. reuteri LR1349 has been deposited at the Bioresource Collection and Research Center (BCRC) of the Food Industry Research and Development Institute (FIRDI) (No. 331, Shih-Pin Rd., Hsinchu City 300, Taiwan, China) under an accession number BCRC 911241 since Aug. 28, 2024, and has also been deposited at the Deutsche Sammlung von Mikroorganismen und Zellkulturen (DSMZ) GmbH under an accession number DSM 35135 since Sep. 9, 2024 in accordance with the Budapest Treaty.

### Example 2. Evaluation of the Efficacy of Limosilactobacillus reuteri Subsp. reuteri LR1349 in Alleviating Blue Light-Induced Retinal Damage

### Experimental Materials:

1. Preparation of Bacterial Suspension of Limosilactobacillus reuteri Subsp. reuteri LR1349:

The Limosilactobacillus reuteri subsp. reuteri LR1349 was inoculated into MRS broth medium (BD Difco™; Cat. No. 288130), and then cultured at 37° C. for 16 hours to 18 hours for activation of the same. After centrifugation at 5000 rpm and 4° C. for 10 minutes, the resultant cell pellet and the supernatant were formed. The supernatant was then collected, and the cell pellet was washed with an appropriate amount of phosphate-buffered saline (PBS), followed by using an appropriate amount of the supernatant to re-suspend the cell pellet (the appropriate amount of the supernatant was also used to adjust bacterial concentration of the subsequently obtained bacterial suspension so as to have a predetermined value), thereby obtaining a bacterial suspension of the Limosilactobacillus reuteri subsp. reuteri LR1349 having a bacterial concentration of 3.3×10⁸ CFU/mL.

### Experimental Procedures and Results:

### A. Administration of Bacterial Suspension and Induction of Retinal Damage:

First, the male ICR mice were randomly divided into 3 groups (n=10 in each group), including one normal control group, one pathological control group, and one experimental group. After that, each of the mice in the experimental group was fed, via oral gavage, with the bacterial suspension of the Limosilactobacillus reuteri subsp. reuteri LR1349 (dose: 1×10⁸ CFU/mouse) obtained in Section 1 of Experimental Materials of this example. Each of the mice in the pathological control group and the normal control group was fed, via oral gavage, with MRS broth medium having a volume equal to that of the bacterial suspension of the Limosilactobacillus reuteri subsp. reuteri LR1349. Each mouse was fed once a day for a time period of 17 weeks.

From the 2^{nd} week after starting administration of the bacterial suspensions or the MRS broth medium, the mice in the pathological control group and the experimental group were subjected to irradiation with blue light having a wavelength of 465±10 nm and an illumination of 108 lux (44.8 μW/cm²) twice a day, 3 hours each time, for a time period of 16 weeks. The mice in the normal control group received no treatment.

### B. Visual Acuity Testing:

At the end of each of the 8^{th} week and the 16^{th} week after starting the irradiation with the blue light, the mice in each group were subjected to visual acuity testing conducted generally according to the method described in Liou J. C. et al. (2018), J. Funct. Foods, 49:314-323. Specifically, each of the mice in each group was placed on a cylindrical platform located in the center of a dark box with screens on four sides thereof, and then gratings respectively having spatial frequencies of 0.437 cycles per degree (cpd), 0.328 cpd, 0.164 cpd, 0.082 cpd, 0.055 cpd, and 0.033 cpd were played on the screens in sequence. Each of the spatial frequencies (i.e., each of the gratings) lasted for 2 minutes, during which such grating moved alternately in clockwise and counterclockwise directions at a rotation speed of 12°/s. Next, the visual acuity threshold was obtained by observing at which of the aforesaid spatial frequency the mice's head could move in the direction of grating movement with an accuracy rate of not less than 70%.

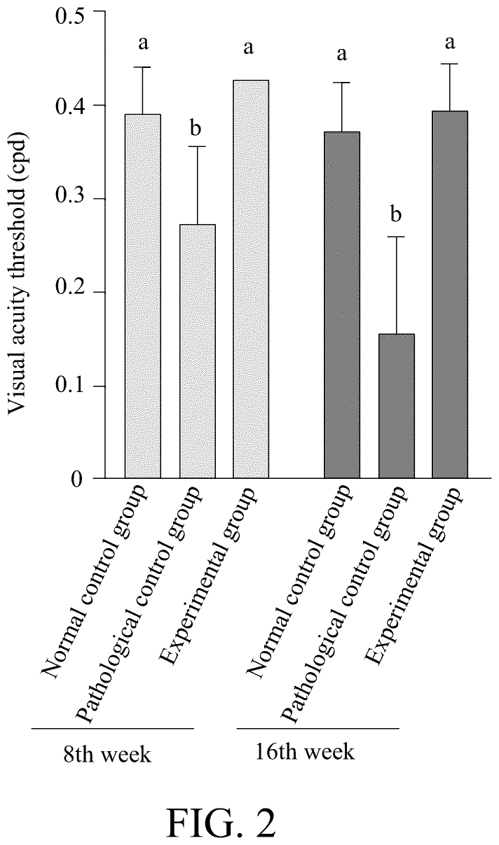

The data thus obtained were analyzed according to the procedures as described in Section 1 of the General Procedures. The results were shown in FIG. 2.

Referring to FIG. 2, at the end of the 8^{th} week after starting the irradiation with the blue light, the visual acuity threshold determined in the pathological control group showed a significant decrease compared with the visual acuity threshold determined in the normal control group, indicating that the blue light successfully induced a decrease in visual acuity in the mice of the pathological control group. In comparison with the pathological control group, the visual acuity threshold determined in the experimental group was significantly increased and was close to the visual acuity threshold determined in the normal control group. At the end of the 16^{th} week after starting the irradiation with the blue light, similar results could be observed. These results show that the Limosilactobacillus reuteri subsp. reuteri LR1349 of the present disclosure can effectively alleviate the decrease in visual acuity caused by blue light irradiation.

### C. Analysis of Electroretinogram (ERG):

At the end of each of the 8^{th} week and the 16^{th} week after starting the irradiation with the blue light, the mice in each group were placed in a dark environment for 12 hours and then anesthetized with Xylazine (dose: 9.75 mg/kg) and Zoletil 100 (dose: 75 mg/kg). Next, each mouse was subjected to mydriasis by instilling one to two drops of 0.5% Tropicamide in a cornea of an eye thereof, and then subjected to analysis of retinal potential using BPM-300 electrodiagnostic system (version of software: 7.0.0.7) (RetinoGraphics) as follows. First, a reference electrode and an indifferent electrode were connected to an outer corner of the eye and a forehead of the mouse, respectively, and a recording electrode was attached to a surface of the cornea. The resistance value of each of the recording electrode and the reference electrode was maintained at 2.1 kΩ, and the resistance value of the indifferent electrode was maintained at 12.1±3.025 kΩ. A white light having a brightness of 3 cd/m² and a flickering frequency of once per second was used to stimulate a retina of the mouse at a distance of approximately 3 mm to 5 mm from the cornea of the mouse, so as to produce retinal potential changes, followed by recording a-wave and b-wave.

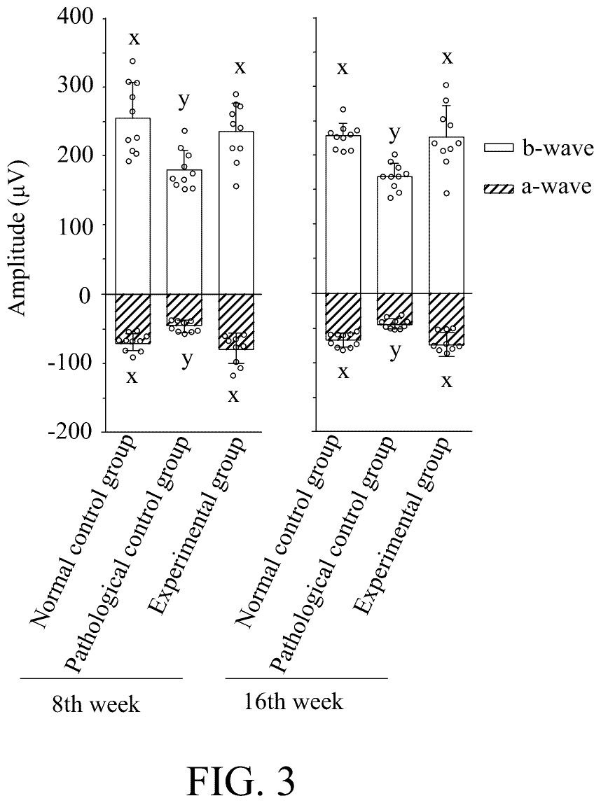

The data thus obtained were analyzed according to the procedures as described in Section 1 of the General Procedures. The results were shown in FIG. 3.

Referring to FIG. 3, at the end of the 8^{th} week after starting the irradiation with the blue light, for each of the a-wave and the b-wave, the retinal potential determined in the pathological control group showed a significant decrease compared with the retinal potential determined in the normal control group, indicating that the blue light successfully induced retinal dysfunction in the mice of the pathological control group. In addition, for each of the a-wave and the b-wave, the retinal potential determined in the experimental group was significantly increased compared with the retinal potential determined in the pathological control group. There was no significant difference between the retinal potential determined in the experimental group and that determined in the normal control group. At the end of the 16^{th} week after starting the irradiation with the blue light, similar results could be observed. These results demonstrate that the Limosilactobacillus reuteri subsp. reuteri LR1349 of the present disclosure can effectively alleviate retinal dysfunction caused by blue light irradiation.

### D. Histopathological Analysis:

At the end of the 16^{th} week after starting the irradiation of the blue light, the mice in each group were anesthetized with 3% to 4% isoflurane and sacrificed, and then eyeballs thereof were removed and subjected to a fixation treatment using a fixative solution (containing 347 mL of ddH₂O, 111 mL 100% acetic acid, 320 ml of 99.5% ethanol and 222 mL of 10% formaldehyde) overnight, followed by a dehydration treatment using ethanol. The dehydrated eyeballs were then embedded with paraffin, followed by slicing, so as to obtain retinal tissue sections each having a thickness of 4 μm.

Thereafter, each of the retinal tissue sections was subjected to hematoxylin-eosin staining using a staining protocol well-known to those skilled in the art, and was then observed and photographed under an optical microscope (Olympus; Model: CX33) at a magnification of 40×, followed by using ViewPoint software to measure the average thickness of an outer nuclear layer (ONL) and an inner segment/outer segment layer (IS/OS) of the retina, and to count the number of nuclei in the ONL.

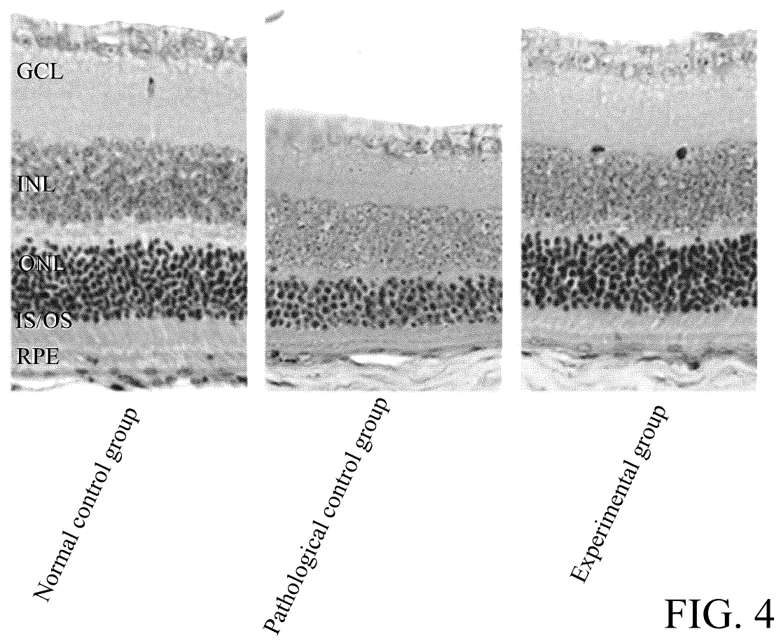

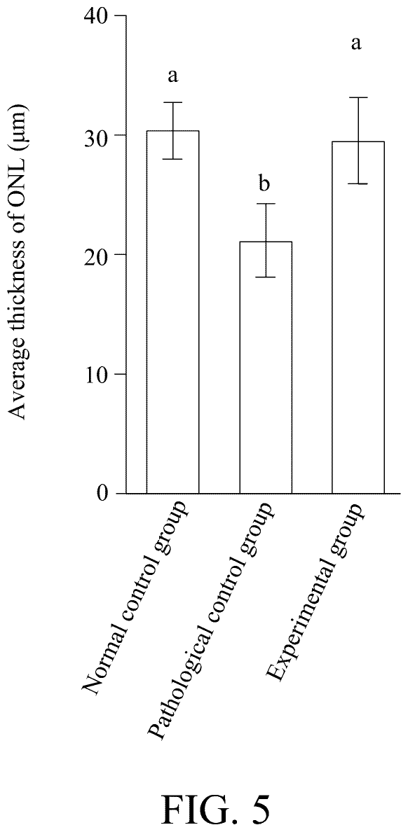

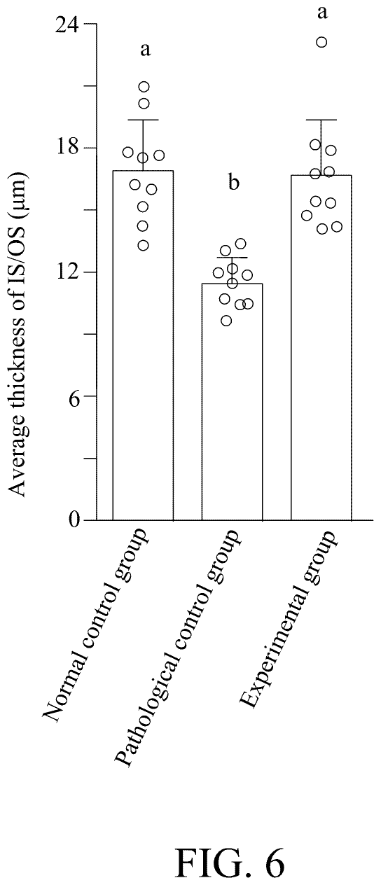

The data thus obtained were analyzed according to the procedures as described in Section 1 of the General Procedures. The results were shown in FIGS. 4 to 7.

Referring to FIGS. 4 to 6, compared with the normal control group, the average thickness of each of the ONL and the IS/OS determined in the pathological control group showed a significant decrease after the irradiation of the blue light, indicating that the blue light successfully induced retinopathy in the mice of the pathological control group. In comparison with the pathological control group, the average thickness of the ONL and the IS/OS determined in the experimental group increased significantly and was similar to that determined in the normal control group.

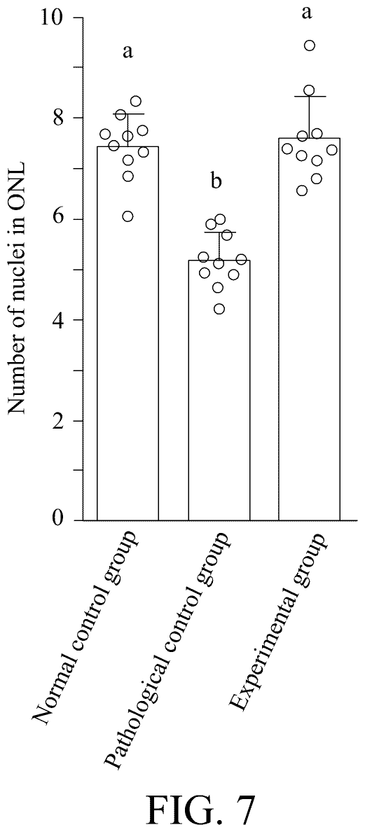

Referring to FIG. 7, compared with the normal control group, the number of nuclei in the ONL determined in the pathological control group was significantly reduced after the mice were subjected to the irradiation of the blue light, indicating that the blue light successfully induced retinopathy in the mice of the pathological control group. In comparison with the pathological control group, the number of nuclei in the ONL determined in the experimental group was significantly increased, and was similar to that determined in the normal control group.

These results demonstrate that the Limosilactobacillus reuteri subsp. reuteri LR1349 of the present disclosure can effectively alleviate the retinopathy cause by blue light irradiation and slow down the loss of retinal tissue.

### E. Immunofluorescence Staining Assay:

A portion of the retinal tissue sections obtained in Section D of this example was subjected to immunofluorescence staining assay using anti-rhodopsin antibody (Abcam; Cat. No. ab98887) as a primary antibody and Alexa Fluor® 488-conjugated goat anti-mouse IgG antibody (Abcam; Cat. No. ab150113) as a secondary antibody according to the manufacturer's instructions. The stained tissue sections thus obtained were observed and photographed using EVOS® FL Auto Imaging System (Thermo Fischer Scientific) at a magnification of 20×, and then fluorescence intensity thereof was quantified using ImageJ software.

The relative fluorescence intensity of each group was calculated using the following Equation:

$$A = B / C$$

where A=relative fluorescence intensity
   B=fluorescence intensity of each group
   C=fluorescence intensity of the normal control group

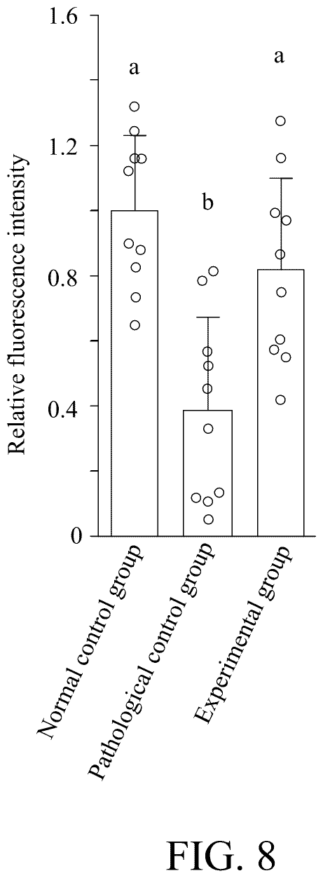

The data thus obtained were analyzed according to the procedures as described in Section 1 of the General Procedures. The results were shown in FIG. 8.

Referring to FIG. 8, compared with the normal control group, the relative fluorescence intensity determined in the pathological control group showed a significant decrease after the irradiation with the blue light, indicating that the blue light successfully caused a large amount of decomposition of photopigments, i.e., rhodopsin, in the retina of the mice in the pathological control group. In comparison with the pathological control group, the relative fluorescence intensity determined in the experimental group was significantly increased and was close to that in the normal control group.

These results show that the Limosilactobacillus reuteri subsp. reuteri LR1349 of the present disclosure can effectively alleviate the retinopathy caused by the irradiation of the blue light, and can slow down the decomposition of the photopigments.

In sum, the Limosilactobacillus reuteri subsp. reuteri LR1349 of the present disclosure can exhibit an excellent effect in alleviating blue light-induced retinal damage.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating blue light-induced retinal damage, comprising administering to a subject in need thereof a composition containing Limosilactobacillus reuteri subsp. reuteri LR1349, which is deposited at the Deutsche Sammlung von Mikroorganismen und Zellkulturen (DSMZ) GmbH under an accession number DSM 35135 in accordance with the Budapest Treaty.

2. The method as claimed in claim 1, wherein the composition is a food product.

3. The method as claimed in claim 1, wherein the composition is a pharmaceutical composition.

4. The method as claimed in claim 3, wherein the pharmaceutical composition further contains a pharmaceutically acceptable carrier.

5. The method as claimed in claim 3, wherein the pharmaceutical composition is in a dosage form selected from the group consisting of an oral dosage form and a parenteral dosage form.


SEQUENCE LISTING (11 sequences)

SEQ ID NO: 1 - DNA, 20 bp, synthetic construct
  source 1..20: mol_type = other DNA; note = forward primer 27F for amplification of 16S rDNA
agagtttgatcctggctcag

SEQ ID NO: 2 - DNA, 18 bp, synthetic construct
  source 1..18: mol_type = other DNA; note = reverse primer 1492R for amplification of 16S rDNA
ggttaccttgttacgact

SEQ ID NO: 3 - DNA, 1406 bp, Limosilactobacillus reuteri
  source 1..1406: mol_type = genomic DNA; note = belongs to the subspecies reuteri
acatgcagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccgcatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattgagttgggcactctagtgagactgccggtgacaaaccggaggaaggtggggacgacgtcagatcatcatgccccttatgacctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagttcgcgg

SEQ ID NO: 4 - DNA, 10 bp, synthetic construct
  source 1..10: mol_type = other DNA; note = primer RAPD-1 for RAPD
ctcaggtcgc

SEQ ID NO: 5 - DNA, 10 bp, synthetic construct
  source 1..10: mol_type = other DNA; note = primer RAPD-A for RAPD
ccgcagccaa

SEQ ID NO: 6 - DNA, 10 bp, synthetic construct
  source 1..10: mol_type = other DNA; note = primer RAPD-B for RAPD
aacgcgcaac

SEQ ID NO: 7 - DNA, 10 bp, synthetic construct
  source 1..10: mol_type = other DNA; note = primer RAPD-C for RAPD
gcggaaatag

SEQ ID NO: 8 - DNA, 10 bp, synthetic construct
  source 1..10: mol_type = other DNA; note = primer RAPD-D for RAPD
gaggacaaag

SEQ ID NO: 9 - DNA, 10 bp, synthetic construct
  source 1..10: mol_type = other DNA; note = primer RAPD-E for RAPD
ggcgtcggtt

SEQ ID NO: 10 - DNA, 10 bp, synthetic construct
  source 1..10: mol_type = other DNA; note = primer RAPD-F for RAPD
ggccacggaa

SEQ ID NO: 11 - DNA, 10 bp, synthetic construct
  source 1..10: mol_type = other DNA; note = primer RAPD-G for RAPD
cgaactagac